(19) United States
(12) Patent Application Publication
(10) Pub. No.: US 2016/0166766 A1
(43) Pub. Date: Jun. 16, 2016

(54) ACOUSTIC BASED DRUG DELIVERY MONITOR

(71) Applicant: OSCILLARI LLC, Bolinas, CA (US)

(72) Inventors: Jeffrey A. SCHUSTER, Bolinas, CA (US); Stephen J. FARR, Orinda, CA (US)

(73) Assignee: OSCILLARI LLC, Bolinas, CA (US)

(21) Appl. No.: 14/903,936

(22) PCT Filed: Jul. 11, 2014
(86) PCT No.: PCT/US14/46367

§ 371 Date: Jan. 8, 2016

Related U.S. Application Data
(60) Provisional application No. 61/845,670, filed on Jul. 12, 2013.

(51) Int. Cl. A61M 5/168; G01F 22/00; A61B 5/00

(52) U.S. Cl. CPC A61M 5/16831; A61B 5/0022; G01F 22/00; A61B 5/7235; A61B 5/742; A61M 2230/40

(57) ABSTRACT

A drug delivery monitoring system comprising a monitor is disclosed that utilizes sound to monitor the occurrence and properties of a drug delivery event. The monitor is affixed to the exterior of a drug delivery device or drug container, and thus does not require disassembly of the drug delivery device and cannot interfere with the operation of the drug delivery device. The monitoring system includes a display device such as a smart phone or tablet computer for analyzing data related to the drug delivery device usage and displaying information to a patient or caregiver before, during, and after a drug delivery event.

### FIELD OF THE INVENTION

The present invention relates to methods and devices for the monitoring of delivery events from a drug delivery device, and displaying data, instructions, and feedback to a patient or care giver.

### BACKGROUND OF THE INVENTION

Many devices exist in the art for delivering drugs to patient. These devices can range from a simple oral capsule to a complex hospital based system. Many technologies currently exist or are disclosed in the art that allow a patient to self administer drugs. These devices include inhalers, autoinjectors, needle free injectors, pumps including patch pumps and bolus pumps, transdermals, sprays, ocular devices, etc.

Many disease states exist wherein available drugs and delivery systems efficaciously treat most patients, but a significant percentage of the patient population are not properly treated due to improper use, or non use, of the drugs and delivery systems. Examples of disease often not correctly treated include, but are not limited to Asthma and Diabetes. Untreated Asthma can lead to expensive emergency room visits, changing to expensive drugs, including biotech proteins such as omalizumab, extreme patient discomfort, or death. Similarly, untreated diabetes can lead to emergency room visits, blindness, nerve damage, cardiovascular events, loss of foot or leg, blindness, or death. Thus, there is an unmet medical need for better means of determining that patients are self administering their medications properly.

Autoinjectors are self contained devices for deliving drugs by injection, either intradermally, subcutaneously, or intramuscularly. Autoinjectors can contain a single dose, or multiple doses, may be disposable or re-fillable, and comprise a self contained power source such as a spring, compressed gas, batteries, or a combustible or pyrotechnic material. Autoinjectors may contain a hypodermic needle, but also may be needle free, jet type injectors. Autoinjectors are often used for chronic conditions where multiple injections must be given in a home setting, for example diabetes, osteoporosis, growth hormone deficiency, and the like. Preferred autoinjectors are multidose, and preferably are dose titratable, for example insulin pens.

Inhalers are devices that allow delivery of drug to the lung, either for treatment of lung diseases such as asthma, chronic obstructive pulmonary disease (COPD), cystic fibrosis, emphysema, chronic bronchitis, pulmonary hypertension, bronchietasis, or for systemic effect for such indications as diabetes, agitation, pain such as migraine pain, post operative pain, cancer pain, or others. Preferred inhaled drugs for systemic effect are those that either currently must be delivered by an invasive means such as injection, or benefit from a more rapid onset than can be achieved with other routes of delivery such as oral.

Some drugs are dosed at prescribed dosing intervals, such as once a month, once a week, once a day, two times a day, etc. Others are dosed when symptoms are present. In either case it is useful to monitor the time and date of delivery events, for example to determine if the patient is complying with the prescribed therapy, or how often they are having symptoms.

Many drug delivery devices require a somewhat complex maneuver to deliver a dose. This is especially true of inhalers, wherein the patient must inhale at a prescribed rate and duration to get optimal delivery. Some require coordination of the dose with the inhalation, although many modern inhalation devices are breath actuated and do not require this coordination. In either case, it is important that the patient continue inhaling for period after the aerosol is generated to ensure the correct dose is delivered. Other actions may be required, such as shaking the inhaler, priming the inhaler, advancing the inhaler to the next dose, removing a cover or cap, or conducting a breath hold after the delivery. Thus there is a value in monitoring parameters of a pulmonary delivery event to determine if the patient is inhaling and conducting other actions in a way that will deliver an optimal dose.

Devices exist for monitoring a disease state, for example in a home setting. Examples include pulmonary function tests such as peak flow and FEV1 meters, blood glucose sensors, and they like. Wireless, for example Bluetooth versions exist that can transmit measured data to a computer, tablet, or smartphone, whereby a record of disease state over time can be displayed. In general, these devices are not capable of also monitoring drug delivery events, and displaying this information together with information related to the disease state.

WO 96/13293 describes a system into which an inhaler can be inserted. A pressure transducer measures the pressure drop in the airflow path and based on a previous calibration, calculates inhalation flow rates and volumes. A microprocessor and on board memory analyze and store the data. A means for triggering the device is provided, which initiates delivery only if a predetermined flow rate is achieved early in the inhalation maneuver. A peak flow meter is also supplied. Pulmonary function data and inhalation profile data are stored with time and date stamp, and can be downloaded by a wired connection for review. The device can supply the user with audio feedback, for example when to next take the drug.

U.S. Pat. No. 7,448,375 describes a device for delivery of insulin by inhalation, wherein better control of insulin delivery is achieved by controlling the volume of air inhaled with the insulin aerosol. Additional features are described, such as a lock out that limits the number of deliveries in a period of time, or a green light that guides the user to inhale at the correct flow rate, said green light transitioning to a red light if the user inhales too rapidly or too slowly.

US 2010/0192948 and similar application WO 2013/043063 disclose a monitor for an asthma inhaler that uses an optical means for monitoring the actuation of the inhaler, and an electronic control module to monitor and store data related to patient usage of the inhaler. An optional audio sensor is included detect sound associated with movement of the medicament container during delivery of a dose and/or sound associated with the inhalation of the medicament by the patient. As disclosed, the audio sensor does not monitor other information such as flow rate or duration. No method is disclosed for how a sound is determined to be a delivery event. No enablement of how the audio sensor is attached to the inhaler is provided.

US 2012/0265548 discloses a system and method for obtaining an indication of an incentive based on the attributes of the individual and a therapeutic component being available to the individual, transmitting the indication of the incentive to a putative provider of the therapeutic component, assigning a component of an incentive partly based on an indication of a therapeutic component administered to a portion of an individual and partly based on a profile of the individual. Incentives may include monetary, service, or other incentives. One disclosed method of acquiring an indication of a therapeutic component being administered is using auditory, visual, or other sensor data of a cover, plunger, button, or other actuator of the dispensing device in operation. Disclosed dispensing devices include an inhaler, syringe, pill dispenser, and transdermal delivery device.

US 2013/0043975 discloses a system and method for determining if a device has been ingested. The device has one or more immersion-responsive structures, mucosal material sensors, pH sensors, and auditory data distillation modules configured to detect one or more of a swallowing sound, a temperature about equal to that of a living body; a pH about equal to that of stomach acid; a pH increase indicative of travel through a small intestine and of earlier ingestion; auditory or optical indicia of ingestion; mucous or mucosa characteristic of an intestine; or an ambient pressure, electrical conductivity, or other device detectable characteristic of immersion in stool or other bodily fluids. Some disclosure of alternative routes of delivery is supplied, including inhalation and injection.

WO 2008/085607 discloses devices for the monitored storage and dispensing of medication, wherein the devices comprise a plurality of storage compartments, wherein each storage compartment has an interior space for storing at least one medication or at least one medication reminder marker; an image capturing device positionable to capture an image of the interior space of each of the plurality of storage compartments; and a communications module for electronically transmitting the image captured by the image capturing device to central monitoring station. The devices may include at least one audio, visual, or tactile means for communicating information, and may include a microphone. The device may further comprise an electronic communications component, including at least one audio, visual, or tactile means, for communicating information from a user of the dispenser to the central monitoring station.

WO 2008/091838 discloses a medicament delivery device such as an auto-injector, a pen injector, an inhaler, a transdermal delivery system or the like which includes an electronic circuit system to track the patient compliance data associated with the use of the medicament delivery device. The device includes an optional an audio output, such as recorded speech, instructing the user in the use of the medical device.

WO 2010/056712 discloses a medicament delivery device such as an auto-injector, a pen injector, an inhaler, a trans dermal delivery system which includes electronic circuit system configured to produce a recorded speech output instructions associated with, for example, stability of the dose for example of a vaccine, an instruction for using the drug delivery device, an instruction for following a regime associated with the drug, and/or a post-delivery instruction. The electronic circuit system configured to produce a signal, such as, for example, a wireless validation signal, when the activation mechanism is actuated.

WO 2011/135353 describes a monitor for an inhalation device wherein a sound transducer is placed inside the device in the air flow path, and inhalation through the device is monitored by measuring the aplitude of the dominant frequency of the sound.

Prior art inhaler devices monitor inhalation flow rate via pressure transducer ports or mechanical means in the inhalation flow path. These means have the problem that they can become blocked or obstructed by foreign objects from the surrounding air, exhaled matter if the patient exhales, coughs, or sneezes through the device, or by drug particles. Thus there is a need for a method of monitoring inhalation parameters in a way that does require a mechanical or pneumatic connection to the air flow path. In addition, these monitoring means and concomitant airway extension have the problem that they may affect the airflow and aerosol properties, changing them from how the device was designed and tested. Thus there is a need for a method of monitoring air flow rate in a way that does not require any modifications to the device airflow path.

Prior art devices monitor the actuation of the device via a means such as electrical or mechanical that interacts with the drug delivery device's actuation and triggering system. This has gives rise to the possibility that a failure of the monitor can lead to a failure of the device, potentially leading to a change in the delivered dose, or no delivered dose. The monitoring system can also change the triggering characteristics of the device, for example requiring a higher triggering force than that which was previously tested in clinical studies. Thus there is a need for a device that monitors the triggering of a drug delivery device without a mechanical or electrical connection to the device actuator or trigger.

Prior art devices were designed to interface either mechanically, electrically, or pneumatically with a specific device in a very specific way. Thus there needed to be a monitor specifically designed for each device. This leads to many difficulties, including the need to develop and maintain a large number of different monitoring systems, and inability to take advantage of economies of scale that would be available if there were a monitoring device that could be used in a generic way with a large number of existing drug delivery technologies, including essentially all inhalation devices.

Prior art devices had to be either factory integrated with the drug delivery system, or assembled in a way by the user that could be somewhat complex and could require partial disassembly of the drug delivery device, giving rise to the possibility of damage to or incorrect assembly of the device. Devices that factory integrated become part of a drug product and thus can be regulated as drug products, often a signicantly higher regulatory hurdle than for a medical device. Thus there is a need for a drug delivery device monitor that can be simply adhered to a drug delivery device using, for example, an adhesive strip or pad with a release liner. Similarly, there is a need for a monitor for drug delivery devices that can be easily attached by a user or care giver that is partially or entirely insensitive to the precise location of the monitor on the device.

### SUMMARY OF THE INVENTION

The current invention is a monitor and monitoring system for a drug delivery device, such as an inhaler or injection device. The invention detects, interprets and thereby monitors the sound made by a drug delivery device when it is, for example, loaded or otherwise prepared, triggered, and drug is delivered, or when air is drawn through an inhaler. Sounds are detected and compared to pre-loaded acoustic waveforms, and the match to these wave forms may be used to identify events, for example triggering of the drug delivery device, or inserting of a dosage form. In addition, identification of an event, for example a triggering event, can prompt the monitoring system to perform additional detection and/or computation, for example determining the duration of drug delivery and thus dose delivered from an auto-injector, or the inhalation flow rate through an inhaler. In addition, the amount of deviation from pre-loaded or previously measured wave forms may be used to rate the quality of an event, for example determining that a dosage form strip has been fully advanced.

The invention is a monitoring system which is comprised of a microphone which translates sound waves into electrical signals. The electrical signals are acquired by a data acquisition system, and provided to a software program within the monitoring system. The monitoring system includes a set of previously recorded or otherwise generated and pre-loaded acoustic waves which correspond to a desired operation of a drug delivery device. The software program within the monitoring system compares sound detected by the microphone with a specific pre-loaded acoustic wave or set of specific pre-loaded acoustic waves and calculates a difference between the detected sound translated into electrical signals and the set of pre-loaded acoustic waves which operate as standards. If this difference falls within a prespecified range, the acoustic wave is identified as an event. Based on this identification, certain events may prompt additional computation, for example of an inhalation flow rate or delivered dose, or other actions, such as prompting the display of a next instruction. These identified events and the result of additional calculations may be provided to the user of the monitoring system. For example, the identification of the opening of a device or removal of a cap may prompt an instruction, for example to load a dose, set a dosage amount, or advance a dose strip. The time, date, identification of an event, computed information such as the dose delivered, and quality of an event such as whether an inhalation was of the correct flow rate and volume, are provided to the user after the event. The inhalation flow rate through an inhalation device is provided to the user via the display in real time during the inhalation, for example as a graph or moving bar or arrow, along with a target inhalation flow rate range. The monitoring system also provides highlighted information to the user when the device has been incorrectly used. For example, with an inhalation device the program can send a prompt indicating that the user should inhale more quickly or inhale more slowly or inhale for a longer period of time during subsequent dosing events.

The monitor of the invention may be a separate device attached to or incorporated inside of a medical or other device, for example a drug delivery device such as an inhaler or injector. However, the monitor of the invention may be comprised of the microphone component the display device, e.g. a smartphone, tablet or laptop computer. This microphone of the display device gathers acoustical information from the use of a medical or other device and translate that acoustical information into an electronic signal. A program downloaded to the display device translates the electrical signal from the microphone into a defined pattern such as a wave pattern, and analyzes and responds as described above. The display can indicate a simple message such as correct use or incorrect use or provide additional information including coaching the user to use the device differently such as by inhaling more quickly, inhaling longer or inhaling more slowly.

The monitor of the invention comprises a microphone, an electronic storage device which holds stored electronic information corresponding to a recorded or otherwise generated acoustic wave related to an acoustic wave generated by a desired operation of a drug delivery device, and a program which compares electronic signals from the microphone detecting an operation of a drug delivery device with the stored electronic information and calculates a measure of the difference. The device may include a screen which displays information relating to the calculated difference. The program may evaluate the calculated difference and identify correct operation of the drug delivery device when the calculated difference is less than a predetermined amount. The program may generate operations and result in displaying instructions, calculating an inhaled flow rate, calculating an inhaled volume, displaying an inhalation flow rate, calculating a delivery dose or other operation generally used in connection with drug delivery devices.

The invention includes a software program which can be loaded onto any computer such as a smartphone, smart watch, computerized glasses, tablet, laptop computer or the like. The program includes a program operation for translating an electrical signal obtained from a microphone into a defined pattern. A standard pattern is stored in a program which standard pattern is associated with sounds generated from the proper use of a given medical device such as a drug delivery device. The program includes a means for comparing the standard pattern with a defined pattern obtained from translating the electrical signal from the microphone. The program also includes an operation for computing a differential between the defined pattern and the standard pattern as well as a means for generating a display of a visual image based on the differential. The program may include operations which generate information for the user of the medical device such as the drug delivery device which is intended to aid the user of the drug delivery device in providing for more consistent drug delivery and treatment of the patient.

The drug delivery device can be any device that makes a measurable sound, for example when a dose is loaded, advanced, prepared, shaken, inhaled, injected, ingested.

In one embodiment, the drug delivery device is a medication inhaler. The monitor can sense actions including but not limited to motion of the inhaler including picking it up or shaking it, removal of a cap or cover, insertion of a unit dose dosage form or multidose reservoir, advancement of a dose strip, metering of a dose from a multidose reservoir, exhalation of the patient prior to delivery, triggering of the inhaler, inhalation rate, duration, and/or volume through the inhaler, or exhalation of the patient after the delivery, for example after a breath hold. The monitoring system can present the patient with information including but not limited to when to dose, reminders to dose, how many doses or what dosage to deliver, which dosage form to use, reminders and training as the proper use of the device including shaking, exhalation prior to use, proper inhalation flow rate and inhalation volume, actual inhalation flow rate and inhaled volume, breath hold reminders and countdowns, and summary information from current and previous drug delivery events. The monitoring system can also incorporate information related to number of remaining doses, expiration due to time since the dosage form was removed from its primary packaging, and/or expiration due to shelf life. The monitoring system may be combined with an additional device such as a pulmonary function meter, and using data from the meter, suggest actions including but not limited to dosing, skipping a dose, and/or dose amount.

In another embodiment, the drug delivery device is a parenteral delivery device, including but not limited to autoinjectors, prefilled injectors, needle free injectors, pumps including patch pumps, bolus pumps, wearable pumps, pole mount pumps, and the like. The monitor can sense actions including but not limited to motion of the injector including picking it up or shaking it, insertion of a unit dose dosage form or multidose reservoir, advancement of a dose strip, metering of a dose from a multidose reservoir, triggering of the injector, removal of a cap or cover, setting of a dose, and/or duration of the delivery. Duration of delivery can be monitored, for example, by listening to the amount of time the delivery takes, e.g. the sound of a motor, the sound of drug flowing through the system, and/or the duration of time from a triggering event to an end event such as a piston hitting a stop. The monitoring system can present the patient with information including but not limited to when to dose, reminders to dose, how many doses or what dosage to deliver, which dosage form to use, reminders and training as the proper use of the device including shaking, cleaning of injection site, injection duration reminders and/or countdowns that remind a patient how long to keep an injection device in place, and summary information from current and previous drug delivery events. The monitoring system may be combined with an additional device such as a blood glucose meter, and using data from the meter, suggest actions including but not limited to dosing, skipping a dose, and/or dose amount.

In another embodiment, the drug delivery device is a container for dosage forms including but not limited to pills, capsules, films, troches, lozenges, pastilles, suppositories, powders, liquids, solutions, suspensions, or unit dose or multidose drug containers designed for use in another drug delivery system including but not limited to inhalers, injectors, pumps, transdermal, nasal systems, sprays including but not limited to sprays for nasal, ocular, dermal, or buccal administration. The monitor can sense actions including but not limited to picking up the container, opening the container, removing the dosage from the container, which well of a multi-well container was opened, and the like. The monitoring system can present the patient with information including but not limited to when to dose, reminders to dose, how many doses or what dosage to deliver, which dosage form to use, which medication to deliver, reminders and training as the proper use of the dosage form including but not limited to shaking, diluting, sucking, swallowing, sprinkling, or dissolving, and summary information from current and previous drug delivery events.

In one preferred embodiment, the drug is a controlled, commonly abused substance, or dangerous substance with high overdose potential, including but not limited to an opioid or other pain medication, alcoholic beverage or other alcohol containing substance, barbiturate, benzodiazepine (particularly alprazolam, lorazepam, and clonazepam), cocaine, or methaqualone. The monitor can sense use of the substance for example by sensing the opening of a container or use of a drug delivery device, and how much is used. The monitoring system can supply a patient or skilled or unskilled caregiver with information related to time since last use, amount used, allowable amount to use, time to next allowed dose, etc. If the usage exceeds allowed amounts, the monitoring system can give a notification to the user or caregiver, or transmit a notification to a person or persons including but not limited to family members, friends, nurses, physicians, poison control centers, emergency medical personnel, or law enforcement authorities. This notification can be sent by any means, such as voice messaging, email, text messaging, ‘tweeting’, and/or posting to a web site. It will be obvious to one skilled in the art that future means of sending notifications will be developed that can be used by the device.

In another embodiment, the drug delivery device is used to deliver a drug to multiple different patients, for example for mass vaccination campaigns, bioterror response, and the like. The monitoring system can monitor the usage of the drug delivery system, give training and feedback to the operator, monitor for correct device operations, measure frequency of dosing, number of doses, location of dosing events, etc.

The monitoring system can also be used with any other device that requires that use or other activity is monitored, must be used a prescribed and/or controlled variable way, and makes at least one sound.

Many drug delivery devices deliver drug from a reservoir, and the amount of drug is controlled by the duration of the delivery. Examples include but are not limited to injectors, infusion systems, pumps, inhalers, nasal delivery systems, transdermal systems, and the like. In one embodiment of the current invention, the monitoring system captures the duration of a sound created by the delivery and/or the time duration between two sounds that is characteristic of the dose delivered, and based on a previous laboratory evaluation of the drug delivery device and optionally the concentration of the formulation, calculates and stores a dose delivered. In a preferred embodiment, the drug delivery device is an autoinjector. In a particularly preferred embodiment, the autoinjector comprises insulin or an insulin analog. Preferably, the monitoring system prompts the patient to continue the injection, for example by keeping a needle, catheter, or the likeinserted or by keeping the autoinjector pressed against the injection site, until the monitoring system determines that the delivery is complete or a predetermined time has elapsed. In another embodiment, the drug delivery device is a bolus injector, and the monitoring system prompts the patient to remove the bolus injector when the injection or infusion is complete. In yet another embodiment, the drug delivery device is a pump, and the monitoring system displays information related to infusion rates, bolussing events, occlusions, device failures, dose remaining, and reminders to change infusion sets, refill, recharge, and/or change batteries.

In a training event, the monitor acoustically acquires a sample wave form, which is stored. In the embodiment wherein the training event is conducted more than once, information related to the variation in the wave form may also be stored. This wave form is then compared with subsequently acquired wave forms, and a goodness of fit algorithm or other method is used to determine if an event has occurred. In one embodiment, the reference wave form is stored on a separate display device. Because this may require that the display device be available, on, and running the software during a dosing event, in a preferred embodiment the waveform is stored on the monitor, and can be transmitted the display device at a later time. The goodness of fit determination can be conducted by the display device, and preferably is, in the embodiment where the reference wave form is only stored on the display device. In a preferred embodiment, the waveform is stored on the monitor, and a goodness of fit determination is made by the monitor as a criterion for storage, and possibly later transmittal to the display device. In a preferred embodiment, the goodness of fit assessment done by the monitor is preliminary, based on a few parameters such as amplitude, duration, etc, and this assessment is used to determine if the wave form should be stored. Subsequently, when connected, preferably wirelessly, to the display device, data are downloaded, and a final goodness of fit determination, and determination of other parameters such as, in the embodiment where the drug delivery device is an inhaler, inhalation flow rate, duration, etc, are performed. In some embodiments, only measured parameters of the event are stored on the monitor and/or display. In a preferred embodiment, the full waveform is stored by the display device, allowing for future changes to the software and/or display configurations.

In one embodiment, the location of the monitor is completely at the discretion of the patient or caregiver, and can be modified based on the patient's preferred method of using the drug delivery device. In a preferred embodiment the patient is given a suggested or required location for attachment of the monitor based on the specific drug delivery device to be used. Depending on the ability of the patient to accurately locate the device, and the sensitivity of the algorithm for identifying an event to the amplitude or amplitudes of the event, the device may or may not require additional calibration.

The monitor may be attached to the drug delivery device, and optionally calibrated, in the factory where the drug delivery device is fabricated. Alternatively, monitors may be attached in a controlled, batch process and optionally calibrated by a third party, for example a pharmacy, hospital, HMO facility, doctor's office, etc. In a preferred embodiment, the monitors are attached individually by the patient or care giver, and optionally calibrated by performing the desired event, such as triggering, inhaling, etc, in a “training mode”.

Any means of attachment can be used, such as fasteners, adhesives, elastic bands, etc. In a preferred embodiment, the attachment is achieved using an adhesive strip or pad, which is supplied with the adhesive covered by a removable release liner. In one embodiment, the adhesive is supplied with a cleaning means, such as a solvent or other cleaning agent infused cotton swab or cloth. In a preferred embodiment, the nature of the adhesive, size and shape of the monitor, and/or the size of the adhesive pad or strip are such that no surface preparation is required. In one embodiment, the monitor is supplied with the adhesive already attached, and ready for use after removal of a release liner. This is preferred for the embodiment wherein the monitor is non-releasably attached to the drug delivery device. In another preferred embodiment, the adhesive pad is in the form of a carrier for the monitor, and the monitor is removably attached to the carrier. In a preferred embodiment, the carrier contains a through hole, and the monitor comprises a mating, raised feature which contains the sound transducer. The monitor raised feature is inserted into the through hole, and is held in place, for example by a friction fit, or preferably by a détente which supplies positive feedback in the form of a click to the user that the monitor is properly inserted. When the carrier is attached to the drug delivery device, the sound transducer is proximate to, or preferably in contact with, the drug delivery device, maximizing the sound level and shielding it from ambient noise. The carrier comprises an adhesive and a release liner, and is non-releasably attached to the drug delivery device. The carrier may be supplied in a single form that is applicable to all expected uses of the monitor, and for example may be flexible, for attachment to rounded or otherwise contoured device surfaces, for example insulin pens. In another embodiment, the carrier may be specific to a device or set of devices, contoured to mate to the surface of the device or devices, and may optionally include a footprint or other fiducial features to aid in proper placement on the device. In one particularly preferred embodiment, the device is a “diskus” device, such as that used by the GSK “Advair” drug product. The diskus has a flat circle approximately 30 mm in diameter, with a raised feature along its circumference, on both the top and the bottom of the device. The carrier may include a portion of its edge which is of the same radius as the raised feature, and can interface with the raised feature. The carrier in this embodiment would have a through hole which is centered on the radius of curvature of the raidiused portion of its edge. In this way, the sound transducer would always be in the same spot at the center of the Diskus flat circle, independent of where on the circumference of the flat circle the edge of the carrier is placed.

The monitor may be supplied with one or more carriers when packaged for sale.

A multiplicity of carriers, possibly different versions depending on the drug delivery device to be used, are preferably also offered for separate sale.

The monitor preferably includes a means for transmitting acquired data to another device for display, analysis, and/or subsequent re-transmission. The transmission to the display device can be by a wired means including but not limited to USB or firewire, but is preferably by a wireless means such as wifi or Bluetooth. It will be obvious to one skilled in the art that future wired and wireless communication protocols and systems will be developed and can be used by the invention. The display device may be a dedicated system supplied with the monitor, but in a preferred embodiment is a device the user already owns, such as a smart phone or tablet. Preferred display devices include but are not limited to mp3 players, smartphones including but not limited to Android phones, iPhones, Blackberry devices, or Microsoft phones, smart watches and other wearable devices, eyeglasses capable of displaying information such as Google glass, tablet, notebook, and desktop computers, automobiles, televisions, and television connected peripherals such as DVD players, Blue-ray players or streamers. It should be noted that electronics technology is rapidly evolving, and it will be obvious to one skilled in the art that related but new display and analysis technologies will be available in the future, and may be used with the current invention.

Preferably the monitor and/or carrier contains features that shield the sound transducer from ambient noise. In a preferred embodiment, the monitor includes noise cancelling technology, which may comprise a second sound transducer which measures the ambient sound, and functionality for subtracting a signal proportional to that sound from the sound signal acquired by the first sound transducer. The constant of proportionality for the subtraction may be a single constant, or may be multiple constants or otherwise contoured based on the sound frequency. The proportionality may be calibrated prior to a delivery event. In one embodiment, the patient is instructed or prompted to hold in position (if required) immediately prior to delivery for a predetermined amount of time, for example with a needle inserted or an inhaler in the mouth, and the ambient sound measured by both transducers is monitored. In this way the noise cancelling can be calibrated in a way optimized for the sound environment in the exact configuration that the delivery will take place.

These data can be used in many ways. In one embodiment, the data may be displayed to the patient in real time, while they are delivering drug, as a method of training and feedback. For example, in the embodiment where the drug delivery device is an inhaler, the patient can be prompted to inhale more slowly or more rapidly, continue inhaling, or trigger the device. In another embodiment, the patient is given feedback on the quality of the delivery maneuver after the delivery event, so they can, for example, improve the maneuver at the next dosing event, or repeat the delivery if it is determined that an insufficient dose was received. The data can also be displayed in tabular form, displaying all of the events available, all of the events in a requested or predetermined interval, all of the events with a particular drug delivery device, etc. These events can be displayed in many ways, such as in tabular form, graphical form, or in a way that highlights incorrectly conducted delivery events. In an additional embodiment, the patient or care giver enters additional data, such as the information related to, for example, the time, date, and severity of a medical event, such as an asthma exacerbation. Such data can also be entered automatically, for example from an electronic medical record. In addition, the data from many patients can be combined and used to determine how well a population uses a drug delivery device, optionally combined with location data such as GPS. These data can be used, for example, in clinical trials, post marketing commitments, or scientific studies, for example to determine which devices are the easiest to use properly, how patients use or mis-use devices. These data can also be used to determine which delivery profiles and compliance result in the best clinical outcomes, and the training and feedback can be modified accordingly

Software for the display system may be supplied with the monitor, but is preferably downloaded by the user from a web site, application store, or the like. Preferably the application allows the user to enter the drug delivery device to be used, and instructions, calibration, images etc. are downloaded that are specific to that device. The software may also allow an option that is generic to any drug delivery or other type of device. In this embodiment, the display unit is used to put the monitoring system in a training mode, and the desired event, for example drug delivery device actuation, is conducted, preferably 1 time, but possibly 2, 3, or more times, while the sound wave form is acquired by the monitoring system. This wave form, or average of multiple waveforms, becomes a reference waveform, and is used to identify subsequent events. While the invention is preferably directed toward drug delivery devices, it can be seen that such a generic system could be used for other applications, such as the ringing of a doorbell or phone, the opening of a refrigerator, medicine cabinet or the like, or any of a number of other applications wherein a list of event times would be useful. The software would preferably comprise locked versions, for example for class II devices and others that require regulatory approval of the software. Open source versions may also be made available for development of optimized applications for lower risk medical and non-medical devices.

In one embodiment, the monitoring system acoustically acquires the reference wave form, which is subsequently stored. In the embodiment wherein the training event is conducted more than once, information relating to the variation in the wave form may also be stored. This reference waveform is then compared with subsequently acquired waveforms, and a comparison algorithm is used to determine if an event has occurred. In one embodiment, the sound information is sent directly to the display device as it is acquired, where it is analyzed, and stored. Because this would require that the display device be available, powered on, and running the software, in a preferred embodiment acquired waveforms are stored on the monitor, and are transmitted the display device at a later time, when the display device and monitor are connected. The goodness of fit determination can be conducted by the display device, and preferably is, in the embodiment where sound information is sent directly to the display device. In a preferred embodiment, the waveform or predetermined characteristics of the waveform of an identified event are stored on the monitor, and a goodness of fit determination is made by the monitor as a criterion for storage. In one embodiment, the reference waveform is stored on the monitor, and the comparison of an acquired waveform to the reference waveform is conducted by the monitor to identify and store events. In a preferred embodiment, the identification of an event done by the monitor is preliminary, based on a few parameters such as amplitude, duration, etc, and this assessment is used to determine if the wave form should be stored. Subsequently, when connected to the display device, data are downloaded, and a final goodness of fit determination, and final determination of other parameters such as inhalation flow rate, dose delivered, duration, etc, are performed. In some embodiments, only measured parameters of the event are stored on the monitor and/or display. In a preferred embodiment, the full waveform is stored by the display device, allowing future reanalysis and display in the case of, for example, future changes to the software and/or display configurations.

Many methods of comparing a measured waveform to a reference waveform can be carried out, including but not limited to calculating a cross correlation and identifying an event based on the height of the cross correlation, calculating a residual sum of squares or other measure of the difference between the sample and reference waveforms, and accepting the sample waveform as an event if the measure of difference is below a threshold value, and comparing the amplitude of certain specified points. These calculations can be carried out in either the time domain or the frequency domain.

In a preferred embodiment, the software is specifically designed for a specific device, formulation, and disease state. This can be done by having a different version of the software available for each combination. In a preferred embodiment, there are one or at most a few versions of the software available, and the device, formulation, and/or disease state are entered by the user using the display device. Based on the selected combination, the display device can select items such as the data to display, sample wave forms, goodness of fit algorithms, parameters to calculate, and optionally where to share the data. For privacy, the user can be prompted to “opt in” to data sharing. The display device may also upload certain information related to the wave form shape, expected amplitude and duration, fit parameters, etc. to the monitor. Optionally, the patient or care giver can customize what data are acquired and/or displayed, and how the data are displayed, and ranges for highlighting a given datum, for example in another color such as red.

Optionally, the patient or care giver may be prompted to enter personal information. This information may include but is not limited to height, weight, body mass index, sex, race, age, disease state, disease severity, and/or pulmonary function parameters including but not limited to vital capacity, peak expiratory flow, and FEV1. Using these input parameters and data from a calibration or training event, relative values measured by the monitoring system may be displayed as absolute values. For example, vital capacity may be known and entered into the device. The patient may then be prompted, during a training event, to exhale fully, and then inhale as deeply as possible through the inhaler, essentially performing a vital capacity maneuver. Based on the duration of the inhalation, and the previously measured vital capacity, an average inhalation flow rate can be computed. Based on this average flow rate, the wave form of the sound during the inhalation, and optionally a physical model that may include corrections for such things as device flow resistance, a calibration of the flow rate vs. measured sound amplitude can be established. Similarly, the patient may be prompted to enter peak expiratory flow or FEV1, and inhale or exhale through the device as rapidly as possible, to establish the calibration. Preferably, if parameters such as pulmonary function parameters are not known at the time of calibration, the device will still operate. In one embodiment, the device uses model predictions of pulmonary function parameters, based on inputted data selected from a list including but not limited to height, weight, body mass index, age, sex, race, disease state and severity. In another embodiment, flow rates and inhaled volumes are displayed as a percentage of the maximum values specific to the patient as determined during a training maneuver. In a preferred embodiment, the data are stored in such a way that actual flow parameters may be computed if pulmonary function parameters are entered at a later date, for example after a visit to a pulmonologist or asthma specialist.

In a preferred embodiment, the inhalation or other flow rate calibration is conducted in a way that is independent of the amplitude of the sound measured during an inhalation event. For example, increasing levels of turbulence may be expected to occur at higher flow rates, which may lead to changes in the frequency domain, for example higher amplitudes in some frequency bands, such as higher frequency bands, relative to other, for example lower, frequencies. Thus by comparing the ratio of amplitudes in two or more frequency bands, the flow rate through the inhaler may be determined based on previous laboratory measurements of the specific inhaler being used, in a way independent of the amplitude of the sound, for example due to variations in placement of the monitor. The spectral parameters may be determined by several methods, including but not limited to band pass filters or Fourier transforms. Subsequently, measurements of the flow rate may be based on spectral measurements, but are preferably based on a calibration of the wave form amplitude performed via the above analysis.

In one embodiment, event waveforms are analyzed using parameters determined during the initial calibration or training, or parameters that are downloaded. In a preferred embodiment, the parameters are recalculated after every event that satisfies the goodness of fit criteria, based on a weighted or unweighted average of a predetermined number of previous events. In this way, if the acoustic wave form, for example of the device triggering, changes over time due to any reason, including but not limited to wear of the mechanical components of the triggering and actuation mechanisms, changes in the acoustic properties of the device due to, for example, drug build up in the airway or changes in formulation volume contained in a drug reservoir, and/or changes over time in the location of the sensor, the waveform standard will evolve accordingly.

In a preferred embodiment, delivery events are stored with a time and date stamp. In the embodiment where the display device must be connected at the time of the event, the time and date stamp can be generated by the display device using its internal clock. In a preferred embodiment where the monitor need not be connected to the display device, the time and date stamp may be generated by the monitor and stored with other data related to the event. In another embodiment, the monitor may have a simple counter such as a seconds counter or an oscillator and counter. The display device when first connected can then determine the time and date corresponding to a given count. In addition, when the display is connected multiple times, the display device can correct for inaccuracies of the monitor counter.

For portability and ease of use, the monitor is preferably battery powered. The monitor may have replaceable or rechargeable batteries or cells. In a preferred embodiment, the batteries have sufficient lifetime as compared with the expected life of the monitor that they need be neither charged nor replaced. In another preferred embodiment, the batteries are integrated with the carrier or adhesive strip, and are changed when the carrier is changed without requiring additional action on the part of the user. The monitor may comprise an additional power source, such as a second battery or a capacitive storage component, or non-volitile memory, to maintain stored events during a battery change or complete battery discharge. It will be obvious to one skilled in the art that novel power sources may be developed in the future that could be used to power the device.

Adhesive may be used to attach the monitor fixedly to a durable drug delivery device. Depending on the lifetime of the drug delivery device, the batteries may need to be replaceable or rechargeable. In a preferred embodiment, the adhesive is used to affix the monitor to a multidose disposable device, such a dry powder inhaler or a metered dose inhaler, or to a multidose disposable component of a durable device, such as a drug cartridge or battery pack. In this embodiment, the monitor may be detachable from the adhesive strip. The monitor may come supplied with multiple adhesive strips, and a new strip may be used to attach the monitor to a new device or drug cartridge. Optionally, the adhesive strip may be integrated with a battery, simplifying use of the monitor by combining the acts of replacing the adhesive strip and replacing the battery. In a particularly preferred embodiment, the monitor is attached unremoveably to a multidose disposable drug delivery device or drug cartridge, the batteries of the monitor do not require replacement or recharging for the lifetime of the disposable drug delivery device or drug cartridge, and the monitor is disposed of with the device or cartridge.

The monitor includes a sound transducer (i.e. a microphone) for acquiring the sample wave forms. While in general the sound transducer can be located anywhere in the monitor, in a preferred embodiment, the sound transducer is associated with the adhesive component, and when the adhesive is applied to a surface of the drug delivery device, the sound transducer is held in contact to that surface by the adhesive. For example, the adhesive may be an adhesive strip or pad containing a hole which contains the sound transducer. In this way, the transducer can be made more sensitive to sounds made by the drug delivery device, and less likely to get a false positive or other interference from external sounds.

In order to prolong battery life and reduce the possibility of false positive event identifications, the monitor has a means for turning it off and on. In one embodiment, the monitor includes a simple on/off switch. In another embodiment, the monitor is turned on and off using commands from the display device. In a preferred embodiment, the monitor is powered off after a predetermined interval of inactivity, either by the display device, or preferably by the monitor itself. In a particularly preferred embodiment, the monitor incorporates a motion sensor such as an accelerometer, and dedicated, low power circuitry that is capable of powering on the balance of the monitor electronics when a motion is sensed, for example when the drug delivery device is picked up, and the monitor turns itself off based on a period of inactivity, the inactivity being determined based on a combination of such parameters as device motion, measured sound amplitude, successful completion of event identification, successful transmission of data to the display device, etc.

The monitor and/or display may incorporate a feedback system to guide the user to the correct delivery maneuver during the delivery event. For example, the patient may be presented with a green and red light on the monitor, or a similar green or red shape on the display. When the patient is inhaling too slowly, the lights do not light up. In one possible embodiment, when the patient inhales too rapidly, the red light flashes, indicating that the patient should inhale slower. A solid green light indicates a proper inhalation. No light indicates a too slow inhalation. Any number of other feedback methods, including but not limited to sounds, graphical displays, or voice instructions, may be used. In one preferred embodiment, the patient is presented with a graphical display of flow rate on the display device. A preferred flow rate range is highlighted, for example by a different color or by a box. When the patient inhales through the inhaler, the flow rate is displayed on the graph. The flow rate may be displayed as a graph of flow rate vs. time, but preferably only the flow rate at the current time point is displayed, for example as a line, box, or arrow on the graph. In this way, the patient can modify his or her inhalation flow rate by inhaling harder or softer until the indicated flow rate is within the preferred range. This feedback may be used for each delivery event. In a preferred embodiment, the feedback method is used during initial training. If the monitor system determines that a delivery event has occurred outside of a prescribed range, the display may prompt the user to use the feedback method again for the next event.

The monitor electronics must conduct many functions selected from a list including but not limited to sound measurement, analysis, storage, wireless transmission, battery management and status, motion sensing, noise cancellation, timing, time and date stamping, power on and off, control of feedback features, storage of sample wave forms and analysis parameters. These features may be implemented using discrete electronic circuits, but are preferably implemented using one or more integrated circuits. In a preferred embodiment, the electrical components consist essentially of a battery, one or more sound transducers, and a single application specific integrated circuit. In another preferred embodiment, the application specific integrated circuit is comprised of one or more sound transducers.

The data generated by the monitor and display device can be used in many ways, including but not limited to dosing reminders, compliance monitors, dose counters, feedback and/or training as to the proper use of the drug delivery device, determining the best way of using the device, drug usage diaries, dosing lock-outs, overdose warnings, alerts to the patient, caregiver, family, legal authorities, etc. The data may also be pooled with the data from other users.

It is an object of the invention to supply a system for monitoring the use of a drug delivery device.

It is a further object of the invention to supply a device which identifies the usage of a device based on the characteristic sound of said usage

It is a further object of the invention to supply a drug delivery device monitor which does not require any electrical, pneumatic, or mechanical interface or interference with the fluid flow, triggering or actuation mechanism of the drug delivery device

It is a further object of the invention to supply a drug delivery device monitor that can be attached to a drug delivery device without requiring any disassembly and reassembly of the drug delivery device.

It is a further object of the invention to supply a monitoring system for an inhalation drug delivery device that is capable of recording drug delivery events and associated inhalation parameters selected from a list including but not limited to inhalation flow rate, depth of inhalation, inhaled volume after the device is actuated, coordination of the inhalation and actuation of the device, inhalation rate and inhaled volume at the time of actuation, etc., without requiring modification of the device airflow paths by the inclusion of, for example, airway extensions, optical, pressure, or other sensors in fluid contact with the device airflow, or holes in the device airway walls, for example for pressure ports.

It is a further object of the invention to supply a means for determining the dose delivered from a drug delivery device by sensing the duration of the sound made by the delivery.

It is a further object of the invention to minimize the abuse potential of addictive or abused drugs by monitoring their usage and notifying predetermined people if the usage is outside of predetermined guidelines.

It is a further object of the invention to ensure that a patient keeps a parenteral delivery device in place until the delivery is complete

It is a further object of the invention to supply a monitor for a drug delivery or other medical device that is used to treat multiple patients, for example in mass vaccination campaigns or bio-terror response.

It is a further object of the invention to supply a means for displaying information such as the time and date of a delivery event, for example on a smart phone or tablet computer.

It is a further object of the invention to supply a monitoring system which can detect an event such as the actuation of a drug delivery device based solely on the sound of the event.

It is a further object of the invention to supply a device which is capable of measuring inhalation parameters, including but not limited to inhalation flow rate, depth of inhalation, inhaled volume after the device is actuated, coordination of the inhalation and actuation of the device, inhalation at the time of actuation, based solely on the sounds made by the actuation of an inhaler and the sound made by the air flowing through the inhaler

It is a further object of the invention to supply a monitor for a drug delivery device that can be easily and quickly attached to the device, for example by the user.

It is a further object of the invention to supply an acoustic monitor for a drug delivery device that is insensitive to the location of the monitor on the drug delivery device.

It is a further object of the invention to supply a method of calibrating a flow monitor for a drug delivery device that does not require any additional flow generation or measuring equipment

It is a further object of the invention to supply a device which uses the sound generated by inhalation through a device to control a means of giving feedback to the patient as to their inhalation maneuver during the drug delivery event.

It is a further object of the invention to supply skilled care givers and facilities with a record of medical device usage to show that prescribed therapies and procedures were delivered in the way intended.

It is a further object of the invention to supply skilled care givers and facilities with a record of when prescribed therapies and procedures were not delivered as intended

It is an object of the invention to reduce or eliminate the possibility of interference to the acoustical signal from ambient noise sources.

It is an advantage of the invention that it has reduced likelihood of damaging or otherwise impacting the functionality of the drug delivery device during installation of the monitor and use.

It is an object of the invention to supply training to the user of a drug delivery device, and suggest that they repeat training if the device is subsequently used incorrectly.

It is an object of the invention to supply instructions during the use of a drug delivery device

It is an object of the invention to supply feed back to a user during a drug delivery event, guiding them to the proper use of the drug delivery event.

It is an object of the invention to minimize or eliminate the need to charge or change the batteries of a drug delivery device.

It is an object of the invention to supply a monitor for a drug delivery device that can be removably attached to the drug delivery device.

It is an object of the invention to supply a system which combines a drug delivery monitor with a monitor of disease state, and to create a single data set containing data from both monitors.

It is an object of the invention to supply a system for pooling drug delivery usage data from many patients to thereby improve patient care.

It is an object of the invention to supply a single monitor that can be used with multiple different drug delivery technologies.

It is an object of the invention to improve morbidity and mortality by ensuring that medications are delivery properly.

It is an object of the invention reduce costs associated with untreated disease due to in-correct delivery or non-delivery of prescribed medications.

It is an advantage of the invention that there is reduced likelihood of interfering with the triggering, actuation, airflow, or aerosol generation of a drug delivery device, and therefore reduced risk of overdose, underdose, or no dose to the patient.

It is an advantage of the invention that it is less sensitive to the location of an acoustic monitor for a drug delivery device.

It is an advantage of the invention that the monitor is easier to install on the drug delivery device.

It is an advantage of the invention that it can respond to a slowly changing acoustic waveform due to wear of device components, depletion of formulation in the drug reservoir, residual drug left on device surfaces, etc.

An aspect of the invention is a monitoring system for use with a drug delivery device, comprising:

a display device;

a monitor comprising an audio sensor;

an adhesive for attaching the monitor to the drug delivery device;

a wireless transmitter for transmitting data from the monitor to the display device.

In another aspect of the invention the monitor is designed to be attached to the drug delivery device after the drug delivery device is fully assembled.

In another aspect of the invention the monitor is designed to be attached to a system that makes sounds when an event occurs, the monitor is designed to acquire a sample of the sound made during the event, and the monitoring system is designed to identify events based on a comparison to the sample.

In another aspect of the invention the attachment of the monitor does not require any disassembly of the fully assembled device.

In another aspect of the invention the monitor does not touch any moving elements of the drug delivery device.

In another aspect of the invention the drug delivery device is an inhaler, and monitor does not change the air flow path of the device.

In another aspect of the invention the adhesive comprises an adhesive pad or an adhesive strip.

In another aspect of the invention the monitor is attached to the drug delivery device in a factory, doctor's office, or pharmacy.

In another aspect of the invention the monitor is attached after the device has been purchased.

In another aspect of the invention the monitor is attached by the end user.

In another aspect of the invention the monitoring system comprises a display device chosen from a smart phone, mp3 players, smartphones, Android phones, iPhones, Blackberry devices, Microsoft phones, eyeglasses capable of displaying information such as Google glass, a smart watch, a wearable device, tablet, notebook computer, desktop computer, television, DVD player, Blue-ray player, or streamer.

In another aspect of the invention the monitoring system comprises a software program.

In another aspect of the invention the monitoring system comprises a downloadable application.

In another aspect of the invention the monitoring system comprises a software package the operation of which can be customized based on a selected drug delivery device that the monitor is attached to.

In another aspect of the invention the monitoring system comprises a software package the operation of which can be customized based information supplied by the user.

In another aspect of the invention the monitoring system comprises a software package that can be customized based on parameters selected from the drug delivery device, the drug, the disease being treated, the state of the disease, properties of the patient selected from height, weight, sex, age, body mass index, race, medical conditions, pulmonary function parameters selected from peak flow, inspiratory flow rate, vital capacity, tidal volume, FEV1, FEVn, local weather conditions, ambient temperature, ambient pressure, location for data sharing, opt in state for data sharing, physician, hospital, country.

In another aspect of the invention the monitoring system comprises a display which instructs a user or a caregiver as to the proper location for attaching the monitor on a selected device.

In another aspect of the invention the monitoring system comprises a display which instructs a user or a caregiver as to the proper procedure for attaching the monitor on a selected device.

In another aspect of the invention the monitoring system comprises a display for giving feedback to the user related to the correct use of the drug delivery device.

In another aspect of the invention the drug delivery device is selected from an autoinjector, a needle free injector, a bolus injector, and an infusion system and further wherein the monitoring system comprises a display device which prompt the user maintain the placement of the drug delivery device until the drug delivery event is completed.

In another aspect of the invention the drug delivery device is an inhaler, and the monitoring system comprises a mechanism for giving feedback and training to the user during a delivery event selected from reminders based on errors made in previous dosing events, a reminder to shake the device, a reminder to fully exhale prior to inhaling, target inhalation flow rate range, actual inhalation rate, a target inhaled volume, actual inhaled volume, when to trigger the inhaler, when to begin inhaling, when to stop inhaling, breath hold duration.

In another aspect of the system the drug delivery device is an inhaler, and the monitoring system comprises a mechanism for giving feedback and training to the user following a delivery event selected verification the device was shaken, actual inhalation flow rate profile, actual inhalation flow rate range, actual inhalation rate a the time the device was triggered, average inhalation flow rate after the start of aerosol generation, actual inhaled volume, actual inhalation duration.

In another aspect of the invention the drug delivery device is an inhaler, and the monitoring system comprises software that determines inhalation flow rate through the inhaler based on the sound made during inhalation.

In another aspect of the invention the drug delivery device is an inhaler, and the monitoring system comprises software that determines inhalation flow rate through the inhaler based on properties of the sound made during inhalation selected from sound volume, sound spectrum.

In another aspect of the invention the drug delivery device is an inhaler, and the monitoring system comprises software that determines inhalation flow rate through the inhaler based on a comparison of the amount of sound in two or more frequency bands and a comparison to a model of the sound in the two or more frequency bands, said model being based on data previously generated using another example of the drug delivery device.

In another aspect of the invention the drug delivery device is an inhaler, and the monitoring system comprises software that determines inhalation flow rate through the inhaler based on properties of sound made during inhalation and additional information selected from a model of sound vs. inhalation rate for the drug delivery device, data from a previous inhalation through the device by the patient, data related to the patient selected from height, weight, sex, age, race, body mass index, vital capacity, peak inspiratory flow rate, inspired volume, tidal volume, FEV1, FEVn.

In another aspect of the invention the monitoring system comprises a data display and software, wherein the data display must be in wireless contact with the monitor and running the software for the monitoring system to operate.

In another aspect of the invention the monitoring system comprises a data display and software, wherein the data display need not be in wireless contact with the monitor and running the software for the monitor to operate.

In another aspect of the invention the monitoring system comprises a data display and software, wherein the monitor is capable of identifying a drug delivery event without the data display in wireless contact with the monitor and running the software, further wherein information related to the drug delivery event is transmitted to the data display at a later time when the data display is in wireless contact with the monitor and running the software.

In another aspect of the invention the monitoring system comprises a data display and software, wherein the monitor is capable of making a preliminary identification of a drug delivery event without the data display running the software, further wherein information related to the drug delivery event is transmitted to the data display at a later time when the data display is running the software, after which transmitting the software makes a final determination of the identification of a drug delivery event.

In another aspect of the invention the monitor comprises software that allows the monitor to acquire a sample audio waveform.

In another aspect of the invention the monitoring system comprises software that compares an acquired audio waveform to a previously stored audio waveform to determine if a drug delivery event has occurred.

In another aspect of the invention the monitor acquires a waveform based on preset criteria, and subsequently sends the waveform to a display unit for further processing.

In another aspect of the invention the monitor comprises batteries which are rechargeable.

In another aspect of the invention the monitor comprises batteries which are replaceable.

In another aspect of the invention the monitor comprises batteries which are neither rechargeable nor replaceable.

In another aspect of the invention the monitor is attached essentially non-removably to the drug delivery device.

In another aspect of the invention the monitor is attached removably to the drug delivery device.

In another aspect of the invention the monitor comprises an electronic component, and a separate component comprising elements selected from:

an adhesive,

a release liner,

a power source

In another aspect of the invention the monitor is supplied as a kit which comprises an electronic component, and a multiplicity of separate components comprising an adhesive, a release liner, and a mechanism for removably attaching one of the separate components to the electronic component, the attachment comprising at least one of: an electrical attachment, a mechanical attachment.

An aspect of the invention is a method, comprising:

downloading software to a display device;

running the software;

instructing a user to select a drug delivery device in the software

instructing the user as to the attachment of a monitor to the drug delivery device;

instructing the user as to the correct usage of the drug delivery device;

providing the user with feedback related to a dosing event.

These and other objects, advantages, and features of the invention will become apparent to those persons skilled in the art upon reading the details of the devices and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 shows one embodiment of the display of the invention.

FIG. 2 shows one embodiment of a monitor and a carrier of the invention.

FIG. 3 shows one embodiment of an adhesive pad and battery of the invention.

FIG. 4 shows a side view of an embodiment of the adhesive pad and battery of FIG. 3.

FIG. 5 shows a two component embodiment of the monitoring system of the current invention including a display device and monitor attached to an inhaler.

FIG. 6 shows a three component embodiment of the monitoring system of the current invention, with a display, a disease state monitor, and the monitor attached to an autoinjector.

### DETAILED DESCRIPTION OF THE INVENTION

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Monitor: An electronic device that is capable of monitoring the sounds made by an event, and transmitting information related to the event to a display device for display, further analysis, and further transmission. The monitor includes a system for adhering it, removably, or non removably, to a drug delivery system. Preferably the transmitting is wireless.

Monitoring System: a system for monitoring the usage of a drug delivery system that has functions selected form providing instructions and/or suggestions to the user, storing, analyzing, and/or displaying data related to usage, sending alerts, monitoring disease states. The Monitoring system of the current invention comprises a sound transducer for monitoring and characterizing events. The Monitoring system preferably comprises systems selected from a monitor, one or more processing systems, a data transmission system which may be wired but is preferably wireless, a display system, and an alerting system. In a preferred embodiment, the monitoring system comprises a monitor with sound acquisition technology, processing and storage functionality, a wireless transmission system, and a mechanism for either removably or permanently attaching the monitor to a drug delivery device. In the preferred embodiment, the monitoring system also includes a display device that comprises a wireless transmission system, processing and storage functionality, display functionality, and alerting functionality. The monitoring system may also incorporate or be interfaced with a disease state monitoring system, including but not limited to a glucose meter or a pulmonary function meter.

Carrier, adhesive strip, and the like: a component that adheres a monitor to a drug delivery or other device. The adhesive strip may be a simple adhesive that adheres the monitor in an essentially non-removable manner In another embodiment, the carrier is attached non-removably to the device, and the monitor is attached removably to the carrier. The carrier preferably comprises an adhesive region which is covered prior to use by a release liner. Preferably the carrier comprises a hole into which an elevated portion of the monitor is secured, preferably by a detente that gives the user feedback that the device is inserted properly. The elevated portion contains a sound transducer, and the carrier and hole are designed such that the sound transducer is in close proximity to or in contact with the surface of the device.

Display device: A device capable of receiving wireless transmission from a monitor, analyzing the transmitted data, and displaying the data. Display devices may include any device capable of supplying the above functionality. Display devices may be purpose built for the application, but are preferably devices that the user already has. Examples of display devices include but are not limited to smart phones, mp3 players, Android phones, iPhones, Blackberry devices, Microsoft phones, eyeglasses capable of displaying information such as Google glass, smart watches, and other wearable devices, tablets, notebook computers, desktop computers, televisions, DVD players, Blue-ray players, or video streamers. Preferred display devices are smart phones and tablet computers. It will be obvious to one skilled in the art that future devices will be developed that are capable of being used as the display device of the current invention.

Waveform: a set of data containing the pressure oscillations of a received sound signal over time. Often waveforms are related to an event to be tracked by the monitoring system, such as a drug delivery event. Preferred waveforms are the sound a drug delivery device makes when it is loaded, readied for delivery, or triggered, the sound of air travelling through an inhaler when a user inhales through it, and/or the sound or sounds created by an autoinjector or pump during delivery.

Compliance monitor: a device that captures the time and date at which a device, preferably a drug delivery device, is used, preferably along with information related to the proper or improper use of the device.

Feedback: Information given to a user of a device, preferably a patient using a drug delivery device, related to their usage of the device. Feedback may be given while a dosing event is occurring, or may in the form of information and suggestions after the event or multiple events. Preferred feedback includes inhalation flow rate and volume during a dosing event from an inhaler.

Sound transducer, audio transducer, microphone and the like: A device which converts a sound signal into an electrical signal of essentially the same shape (over a range of frequencies) with an amplitude which is proportional to the amplitude of the sound signal.

The terms event, dosing event, delivery event, and the like shall be interpreted to mean an occurrence which is monitored by the monitoring system of the current invention. Preferably the occurrence is the administration of drug to a patient in need thereof, preferably by a drug delivery device, which is preferably but not limited to the intrapulmonary or transdermal route of administration, infusion, or injection. Information related to dosing events is preferably acquired by a monitor and transmitted to a display device.

The term “inspiratory flow rate”, “inspiratory flow” and the like shall mean a value of the volume of air per unit time passing through an inhaler during a dosing event.

The term “inspiratory volume”, “inspired volume” and the like shall mean a measured, calculated and/or determined volume of air passing through an inhaler and into the lungs of a patient

The term “inspiratory flow profile” shall be interpreted to mean data calculated in one or more events measuring inspiratory flow and cumulative volume over time during a delivery event

The term “formulation” is used herein to describe any pharmaceutically active drug by itself or with a pharmaceutically acceptable carrier preferably in a flowable form which is preferably a liquid or powder. Liquid formulations are preferably solutions, e. g. aqueous solutions, ethanolic solutions, aqueous/ethanolic solutions, saline solutions and colloidal suspensions. Formulations can be solutions or suspensions of drug in a low boiling point propellant. Preferred formulations include liquids and powders for inhalation, and liquids for injection.

The terms “lung function” and “pulmonary function” are used interchangeably and shall be interpreted to mean physically measurable operations of a lung including but not limited to (1) inspiratory and (2) expiratory flow rates as well as (3) lung volume. Methods of quantitatively determining pulmonary function are used to measure lung function. Quantitative determination of pulmonary function may be important when delivering analgesic drugs in that respiration can be hindered or stopped by the overdose of such drugs. Methods of measuring pulmonary function most commonly employed in clinical practice involve timed measurement of inspiratory and expiratory maneuvers to measure specific parameters. For example, forced vital capacity (FVC) measures the total volume in liters exhaled by a patient forcefully from a deep initial inspiration. This parameter, when evaluated in conjunction with the forced expired volume in one second (FEV1), allows bronchoconstriction to be quantitatively evaluated. A problem with forced vital capacity determination is that the forced vital capacity maneuver (i.e. forced exhalation from maximum inspiration to maximum expiration) is largely technique dependent. In other words, a given patient may produce different FVC values during a sequence of consecutive FVC maneuvers. The FEF 25-75 or forced expiratory flow determined over the midportion of a forced exhalation maneuver tends to be less technique dependent than the FVC Similarly, the FEV1 tends to be less technique dependent than FVC Similarly to FEV1, FEVn is the forced expiratory volume in n seconds. In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest air flow rate in liters per minute during a forced 15 maximal exhalation, is well correlated with overall pulmonary function in a patient with asthma and other respiratory diseases. The present invention carries out treatment by administering drug in a drug delivery event and monitoring lung function in a monitoring event. A series of such events may be carried out and repeated over time to determine if lung function is improved. Each of the parameters discussed above is measured during quantitative spirometry. A patient's individual performance can be compared against his personal best data, individual indices can be compared with each other for an individual patient (e.g. FEV1 divided by FVC, producing a dimensionless index useful in assessing the severity of acute asthma symptoms), or each of these indices can be compared against an expected value. Expected values for indices derived from quantitative spirometry are calculated as a function of the patient's sex, height, weight and age. For instance, standards exist for the calculation of expected indices and these are frequently reported along with the actual parameters derived for an individual patient during a monitoring event such as a quantitative spirometry test.

### DETAILED DESCRIPTION OF THE INVENTION

The current invention is a monitoring system for a drug delivery device, preferably an inhaler or autoinjector, that monitors the sound made by the device when it is, for example, loaded or otherwise prepared, triggered, when the drug is delivered, or when an inhaler in inhaled through. The measured sounds are preferably compared to pre-loaded acoustic waveforms and the match to these wave forms is used to identify a desired event, such as the loading or triggering of the device.

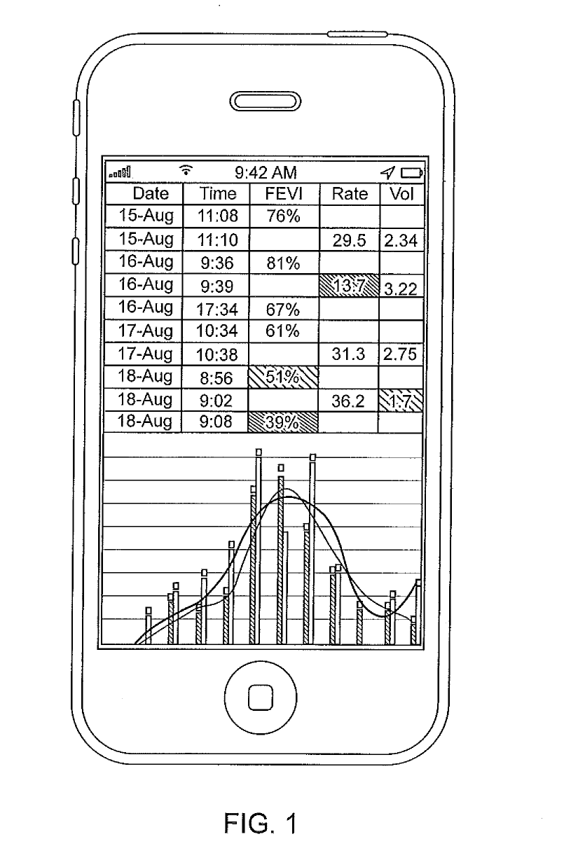

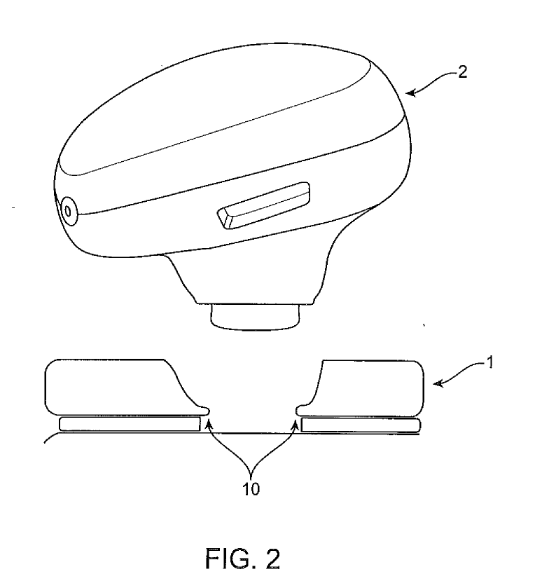

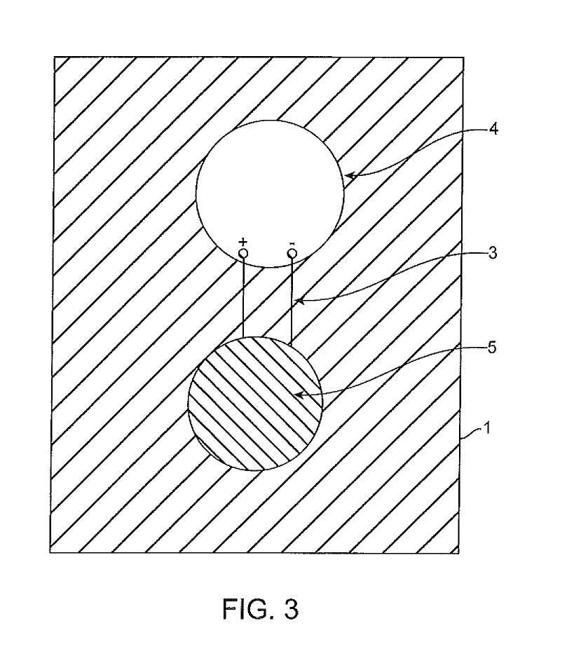

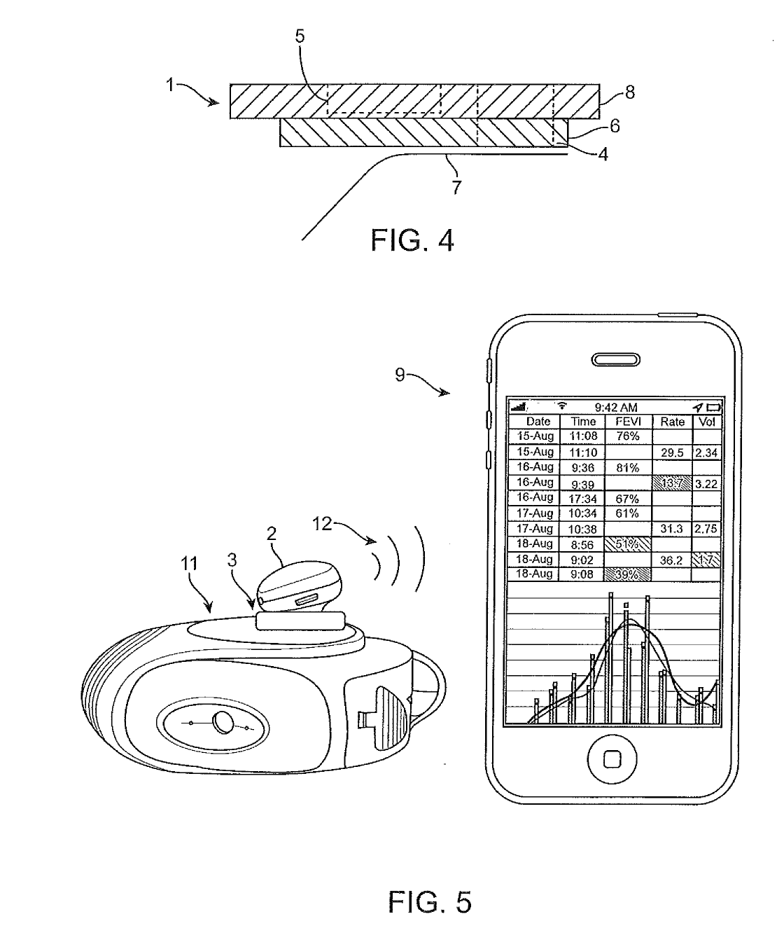

FIG. 1 shows an embodiment of display device 9 of the invention. As shown, display device 9 is a purpose designed display and control device that can only be used with the device. Display device 9 can be any of a number of devices capable of displaying data and sending control commands to monitor 2, including but not limited to smart phones, mp3 players, smartphones, Android phones, iPhones, Blackberry devices, Microsoft phones, eyeglasses capable of displaying information such as Google glass, smart watches, and other wearable devices, tablets, notebook computers, desktop computers, televisions, DVD players, Blue-ray players, or streamers. In a preferred embodiment of the invention, the display device is a smart phone or tablet computer.

FIG. 2 shows an embodiment of the invention wherein monitor 2 is ready to be removably attached to adhesive pad 1. Preferably, in this embodiment of the invention the user is supplied with a monitor 2 and a plurality of adhesive pads 1. Prior to use, monitor 2 is attached to adhesive pad 1 with a click attachment, screw attachment, or bayonet attachment. As shown in FIG. 2, monitor 2 clicks into place in adhesive pad 1 via detents 10. Subsequently, adhesive pad 1 is attached to the drug delivery device to be used, preferably in a predetermined location. When the disposable drug delivery device or disposable drug cartridge of a durable device is expended, monitor 2 is removed from adhesive pad 1, and adhesive pad 1 is disposed of with the disposable device or drug cartridge.

FIG. 3 shows a top view of adhesive pad 1 prior to attachment to monitor 2. Adhesive pad 1 optionally comprises energy source 5 such as an electrical cell or battery. Adhesive pad 1 also has hole 4 into which a mating feature on monitor 2 is inserted. Hole 4 is preferably a through hole so that an audio transducer at the tip of the mating feature of monitor 2 can be brought into close proximity or physical contact with the drug delivery device. Hole 4 optionally contains electrical contacts attached to electrical leads 3 for supplying electrical power to monitor 2. Hole 4 also contains detent features 10 to provide a positive attachment and click when the mating feature on monitor 2 is inserted.

FIG. 4 shows a side view of adhesive pad 1. Substrate 8 supplies mechanical strength to adhesive pad 1, and contains battery 5 and electrical leads 3. Substrate 8 may be rigid, or may be compliant for installation on profiled surfaces, for example the round surface of an insulin pen. Adhesive layer 6 is non-removably attached to substrate 8. Adhesive layer 6 may be thick and compliant enough to conform to non-planar surface profiles. Attached to adhesive layer 6 is removable release liner 7, shown in FIG. 4 partially removed. Hole 4 extends through substrate 8 and adhesive layer 6, but preferably not through release liner 7, so that it is obvious into which end of hole 4 the mating feature on monitor 2 should be inserted.

Use of the embodiment of FIGS. 1-4 is as follows. The system software, for example a smart phone or tablet application, is downloaded into display device 9, and the software is started. Display device 9 prompts the user to enter information selected from a list including but not limited to the drug delivery device to be used, drug information including but not limited to dosing frequency, drug expiration, disease state, prescribing physician, and patient information such as age, height weight, body mass index, race, sex, agreement with sharing of data, etc. The following steps are conducted with instruction from display device 9.

Monitor 2 and one of adhesive pad 1 are removed from their packaging, and a feature on monitor 2 containing the audio transducer is inserted into the hole in adhesive pad 1. Upon making of the electrical connection with leads 3 of adhesive pad 1, monitor 2 powers up automatically, and automatically pairs with display device 9.

Display device 9 then instructs the user to remove the drug delivery device from its packaging, remove release liner 7 from adhesive pad 1, and instructs the user as to the proper placement of adhesive pad 1 on the drug delivery device.

FIG. 5 shows the monitoring system of this embodiment at this stage, wherein monitor 2 is attached to inhalation device 11, and is transmitting wireless signal 12 to display device 9.

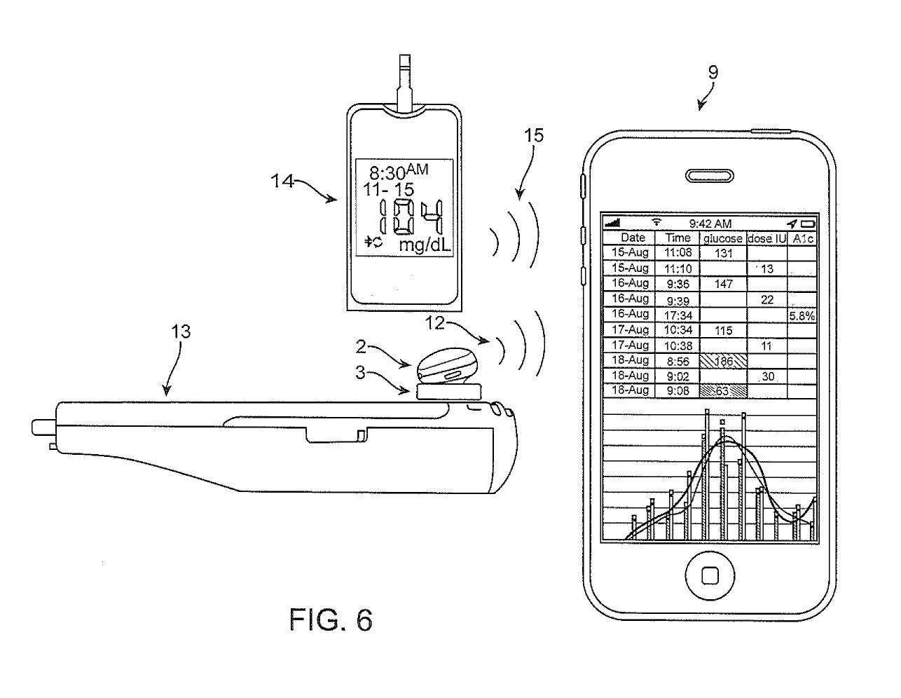

FIG. 6 shows the monitoring system of this embodiment attached to autoinjector 13. Also shown is optional blood glucose monitor 14 and wireless signal from glucose monitor 14 to display device 9.

Optionally, display device 9 instructs the patient in the performance a maneuver, such as a dose delivery or inhalation through an inhaler, in order to train the system, calibrate the sound wave form amplitude, verify functionality, etc.

The user is then instructed in the proper use of the device. Following a dosing event, display device 9 displays information related to the delivery event, and demonstrates to the user how to display information following future events.

Display device 9 continues to supply the user with additional information, for example dosing reminders, doses remaining, suggestions for improving delivery such as inhaling at a different rate or volume, and a record of all dosing events. Optionally display device 9 transmits the dosing data to, for example, the prescribing physician, the drug manufacturer, the drug delivery device manufacturer, or a medical data sharing web site.

When the drug reservoir of the drug delivery device is nearly depleted or the drug is nearly expired, display device 9 prompts the user to replace the device or drug cartridge. Monitor 2 is detached from adhesive pad 1 by pulling in a direction perpendicular to substrate 8. Adhesive pad 1 is disposed of with the drug delivery device or reservoir. A new adhesive pad 1 and drug delivery device or reservoir are removed from their packaging, and the above steps are repeated.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

A physician has prescribed a long acting bronchodilator/inhaled corticosteroid dry powder inhaler product to a patient suffering from asthma, but the patient continues to have asthma attacks. The physician suggests the use of the monitoring system of the current device, and supplies the patient with the results of a recent pulmonary function test for vital capacity.

The patient purchases the monitor from the local pharmacy. Following the directions supplied with the monitor, the patients downloads an associated application to her smart phone, and runs the application.

The application prompts the patient to enter the type of inhaler being used, and her vital capacity. The patient, based on prompts from the smart phone application, removes a release liner from an adhesive pad on the monitor, and applies the adhesive pad to a location on a new inhaler as shown by a picture displayed by the smart phone application. Again following prompting by the smart phone application, the patient pairs the monitor to the smart phone using the Bluetooth functionality of the phone.

Following verbal prompting from the smart phone, the patient exhales as fully as possible, puts the inhaler in her mouth, and inhales as deeply as possible. The monitor recognizes the characteristic sound of inhalation through the device based on criteria wirelessly uploaded by the smart phone application, and wirelessly sends the waveform to the smartphone.

Based on the assumption that the patient inhaled to her vital capacity, and using the sound waveform of the inhalation and laboratory data related to the sound generated by the dry powder as a function of inhalation flow rate, and the fact that the integrated flow rate over the duration of the inhalation must equal her vital capacity, the smart phone application calculates a calibration of flow rate vs. sound amplitude specific to this particular monitor as installed on this device. The inhaler and monitor are now ready to use.

When the patient doses using the device, she is notified by the smart phone app that she inhaled too rapidly, and did not inhale for a sufficient duration to get the entire dose. The application suggests a deeper, slower inhalation, and suggests that she look at the application running on the smart phone the next time she is using the inhaler.

The next day, the patient turns on her smart phone and opens the application prior to using her inhaler. The application recognizes sounds that are characteristic of advancing the dose strip to the next dose, and automatically displays a screen is that graphical representation of inhalation flow rate, with a highlighted target range for flow rate, and a reminder to exhale fully before inhaling through the device. When the patient starts inhaling, she finds she can keep the inhalation rate in the target zone, and receives verbal reminders from the application to continue inhaling. When her inhalation is completed, she is presented with a breath hold countdown timer. She then receives feedback that her inhalation was done correctly.

The next day she uses the smart phone application again, and again is able to achieve a successful delivery. The following day, she feels she can complete the inhalation maneuver without the feedback screen, and does not use the smart phone. She does not receive a notification that there was an issue with the inhalation. Curious, she looks at the log and it shows the day's inhalation as successful.

She continues dosing with the device without thinking about the application.

About two weeks later, she receives a notification that she needs to inhale more deeply. She opens the log in the smart phone application, and it shows that her inhaled volume had been slowly decreasing. The next day, she uses the application feedback function during her inhalation, and thereafter receives no additional notifications of an incorrect inhalation while using that device.

During the third week, she receives a notification that she has forgotten to take her dose, and takes the dose at the next convenient time.

When there are only 5 doses left in her inhaler, she receives a notice that she needs a new one. She calls the pharmacy, and the next day picks up her prescription refill.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A monitoring system, comprising:
a microphone;
an electronic storage device holding stored electronic information corresponding to acoustic waves associated with a desired operation of a drug delivery device;
a program which compares electronic signals from the microphone detecting an operation of a drug delivery device with the stored electronic information and calculates a measure of a difference.

2. The monitoring system of claim 1, further comprising:
a screen which displays information related to the calculated measure of difference.

3. The monitoring system of claim 2, wherein the program evaluates the calculated measure of difference and identifies correct operation of the drug delivery device when the calculated measure of difference is less than a predetermined amount.

4. The monitoring system as claimed in claim 3, wherein the program generates an operation when the calculated measure of difference is less than the predetermined amount and the operation is selected from the group consisting of:
displaying an instruction;
calculating an inhalation flow rate;
calculating an inhaled volume;
displaying an inhalation flow rate; and
calculating a delivered dose.

5. The monitoring system as claimed in claim 4, wherein the system is connected to a drug delivery device.

6. The monitoring system as claimed in claim 5, wherein the drug delivery device is selected from the group consisting of an inhaler and an autoinjector.

7. A monitoring system, comprising:
a microphone;
a set of acoustic waves corresponding to events in the desired operation of a drug delivery device;
a program for comparing sound detected by the microphone with a specific acoustic wave and calculating a measure of a difference, wherein an event is identified when the difference is less than a value which is prespecified for that event; and
a screen for showing the event identified.

8. The monitoring system of claim 7, wherein the event is triggering of the drug delivery device.

9. The monitoring system of claim 7, wherein the identification of the event prompts the program to take an action selected from the group consisting of:
displaying an instruction;
calculating an inhalation flow rate;
calculating an inhaled volume;
displaying an inhalation flow rate; and
calculating a delivered dose.

10. A software program loaded into a smartphone, smartwatch, computer glasses, computer tablet, or laptop computer, the program comprising:
a means for translating an electrical signal obtained from a microphone to a defined pattern;
a standard pattern stored in the program which standard pattern is associated with a proper use of a medical device;
a means for comparing the standard pattern with the defined pattern obtained from translating the electrical signal of the microphone;
a means for computing a differential between the defined pattern and the standard pattern; and
a means for generating a display of a visual image based on the differential.

11. The program of claim 10 wherein the program is loaded into a smartphone device.

12. The program of claim 10, wherein the drug delivery device is selected from
a handheld portable inhaler device
an autoinjector.

13. A monitoring system, comprising:
a monitor comprising a microphone and a wireless transmitting and receiving means;
a carrier;
a display device comprising wireless transmitting and receiving means; and
a program;
wherein the monitor is removably attached to the carrier,
further wherein the carrier is essentially irremovably attached to the drug delivery device.

14. The monitoring system of claim 13, further comprising:
a multiplicity of carriers.

15. The monitoring system of claim 13, further comprising:
a set of acoustic waves corresponding to events in the desired operation of a drug delivery device;
a program for comparing sound detected by the microphone with a specific acoustic wave and calculating a measure of a difference, wherein an event is identified when the difference is less than a value which is prespecified for that event.

16. The monitoring system of claim 15, wherein the identification of the event prompts the program to take an action selected from the group consisting of:
displaying an instruction;
calculating an inhalation flow rate;
calculating an inhaled volume;
displaying an inhalation flow rate; and calculating a delivered dose.
